(19) United States
(12) Patent Application Publication
(10) Pub. No.: US 2018/0021659 A1
(43) Pub. Date: Jan. 25, 2018

(54) AIRWAY AND OXYGEN ENHANCEMENT MOUTHPIECE

(71) Applicant: P3 Athletics Inc., Calgary (CA)

(72) Inventors: Paul Bradley Layzell, Calgary (CA); Russell Tanji, Pearl City, HI (US)

(21) Appl. No.: 15/474,920

(22) Filed: Mar. 30, 2017

Related U.S. Application Data
(60) Provisional application No. 62/315,552, filed on Mar. 30, 2016.

(51) Int. Cl. A63B 71/08; A61M 16/04

(52) U.S. Cl. CPC A63B 71/085; A61M 16/0488; A63B 71/0054

(57) ABSTRACT

The present disclosure relates to systems and methods for identifying a mouthpiece thickness that provides an athlete with optimal breathing that enhances the performance of an athlete when participating in a contact sport. Mouthpieces and methods consistent with the present disclosure may also increase the safety of a contact sport by identifying mouthpiece sizes that correspond to a lower injury rate.

### CROSS-REFERENCE TO RELATED APPLICATIONS

This application is a continuation of and claims priority benefit of U.S. provisional patent application No. 62/315,552 filed Mar. 30, 2016, entitled Oxygen Enhancement Mouthpiece, the disclosure of which is incorporated herein by reference.

### BACKGROUND OF THE INVENTION

### Field of the Invention

The present invention is generally related to mouthpieces. More specifically, the present invention is related to mouthpieces for airway and oxygen enhancement, particularly in relation to significantly reducing the risk of certain concussions and injuries that occur due to a restricted airway, fatigue, to any possible jaw, respiratory, physiological, neurological disorders or deficiencies.

### Description of the Related Art

Modern-day athletes, soldiers, and first responders often wear mouthpieces for tooth and gum protection during high impact or high stress environments. Commonly, these mouthpieces are fabricated from soft thermoplastic, may be form fitted to one arch of the mouth. These standard mouthpieces have a standard thickness in portions between the teeth of a person, they are frequently either smooth or have a predetermined vertical thickness. In most cases, during activity, the standard thicknesses significantly restrict the flow of oxygen which increased fatigue. This increased fatigue in turn impairs neurological function, increases physiological stress, and reduces physiological performance of a person while significantly increasing the risk of injury.

Currently, there are no systems or methods that may be used to identify an preferred or optimal vertical opening that provides an athlete with increased airway and blood oxygenation to the brain when the athlete is engaged in physically or mentally demanding activities. What are needed are proper mouthpieces and systems that can identify preferred proper spacing and incisal opening to optimize oxygen uptake to the brain that are customized to the desired output of an individual user.

Restricting airflow impairs neurological and physiological function. A restricted airway significantly increases the risk of injuries, such as concussions in persons engaged in physical activity. By increasing the opening of an airway of person to a preferred point, risks associated with performing a type of physical activity may be reduced. Methods and systems that best promote preferred incisal openings for particular individuals will, thus increase the performance of those individuals while reducing their risk of concussions and injuries to those individuals.

### SUMMARY OF THE PRESENTLY CLAIMED INVENTION

Embodiments of the presently claimed invention relate to methods, systems, and non-transitory computer-readable storage media for enhancing the airway, performance, recovery, and safety of athletes participating in contact sports and other physically or mentally demanding activities.

A method of the presently claimed invention may collect data for each of a plurality of different mouthpieces when the athlete performs an athletic activity using different mouthpieces over time. Each different mouthpiece may have a different vertical opening, where each different thickness of the different mouthpieces corresponds to a different incisal and occlusal opening in a mouth of the athlete when the athlete wears the mouthpiece. The method may also include analyzing the collected data and identifying, based on the analysis, a preferred mouthpiece of the each of mouthpieces of different vertical opening sizes.

When the method is implemented in a non-transitory computer readable storage medium, the method may also collect data for each of a plurality of different mouthpieces when the athlete performs an athletic activity using different mouthpieces over time. Here again, each different mouthpiece may have a different thickness, where each different thickness of the different mouthpieces corresponds to a different incisal opening in a mouth of the athlete when the athlete wears the mouthpiece. The method may also include analyzing the collected data and identifying, based on the analysis, a preferred mouthpiece of the each of mouthpieces of different vertical opening sizes.

A system of the presently claimed invention may include a plurality of different mouthpieces where each mouthpiece has a different thickness, where the different thickness of each of the plurality of different mouthpieces corresponds to a different incisal opening in a mouth of the athlete when the athlete wears the mouthpiece. Data for each of a plurality of different mouthpieces may be collected when the athlete performs an athletic activity using each of the different mouthpieces over time. The collected data may then be analyzed and a preferred mouthpiece of the plurality of mouthpieces of different sizes may be identified based on the analysis.

### BRIEF DESCRIPTION OF THE DRAWINGS

FIG. 1 illustrates a mouthpiece in a perspective view.

FIG. 2 illustrates the mouthpiece of FIG. 1 in a side view.

FIG. 3 illustrates a partial cross-sectional view of a mouthpiece in the mouth of a person.

FIG. 4 illustrates a partial cross-sectional view of a placebo mouthpiece in the mouth of a person.

FIG. 5 illustrates a partial cross-sectional view of a mouthpiece in the mouth of a person.

FIG. 6 illustrates an exemplary flow chart of steps that are consistent with a method of the present disclosure.

FIG. 7 illustrates an exemplary flow chart of a method for analyzing the effectiveness of players using a mouthpiece versus those same players using a placebo.

FIG. 8 illustrates an exemplary flow chart of a method that identifies optimal incisal openings for an athlete.

FIG. 9 illustrates an exemplary method for cross referencing incisal openings with player risk factors.

FIG. 10 illustrates an exemplary insurance method consistent with the present disclosure.

FIG. 11 illustrates an exemplary method of the present disclosure applied to fantasy football.

FIG. 12 illustrates an exemplary method from monitoring long term versus short term performance and safety metrics relating to athletes wearing airway and oxygen enhancement mouthpieces.

FIG. 13 illustrates an exemplary method of the present disclosure where performance and safety metrics are collected when athletes play different positions in a game or series of games.

FIG. 14 illustrates a block diagram of a computing device that may be used to implement various embodiments of the present invention.

### DETAILED DESCRIPTION

The present disclosure relates to systems and methods for identifying a preferred or proper incisal opening and mouthpiece thickness that provides an athlete with optimal breathing that enhances the performance of an athlete when participating in a contact sport. Mouthpieces and methods consistent with the present disclosure may also increase the safety of a contact sport by identifying mouthpiece sizes that correspond to a lower injury rate.

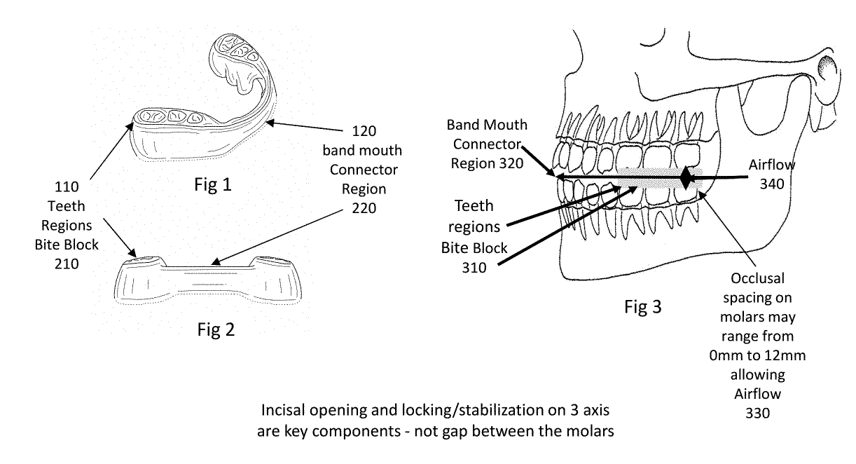

FIG. 1 illustrates a mouthpiece in a perspective view, and FIG. 2 illustrates the mouthpiece of FIG. 1 in a side view. The mouthpieces of FIGS. 1 and 2 include teeth regions of a bite block 110/210 in these mouthpieces. Recessions that align with the teeth of an athlete are shown as tooth shaped recessions in FIG. 1. FIGS. 1 and 2 also include a band mount connector region 120/220 on a front surface of the mouthpiece. This band mount connector region 120/220 may have a plastic band (not depicted) attached to it that attaches to a helmet. Such bands help keep an athlete from losing the mouthpiece or allowing the referee or umpire to visually see a mouthpiece is being used.

FIG. 3 illustrates a partial cross-sectional view of a mouthpiece in the mouth of a person. FIG. 3 also includes a band mouth connector region 320 and a bite block with teeth regions 310. Note that the mouthpiece of FIG. 3 provides an incisal opening where the mouthpiece is locked and stabilized in three directions. By maintaining an incisal opening that is optimized for an athlete, the mouthpiece provides an unobstructed airflow 340 that best allows an athlete to breathe efficiently. FIG. 3 illustrates an incisal opening that may be 3 millimeters (mm), for example. The optimized incisal opening can range from 0.5 mm to 6.0 mm or more depending on the musculoskeletal parameters and desired output, this helps increase the oxygenation of the brain and muscles of an athlete during and after the athlete performs an activity. By optimizing oxygenation levels, risks associated with brain injuries, fatigue, oxygen deprivation, stress, anxiety and dehydration may be reduced. Note that the mouthpiece illustrated in FIG. 3 sets an occlusal spacing 330 that can range, for example, between 0 mm to 12 mm. This is because the bite block 310 of FIG. 3 prevents a person from closing their mouth completely.

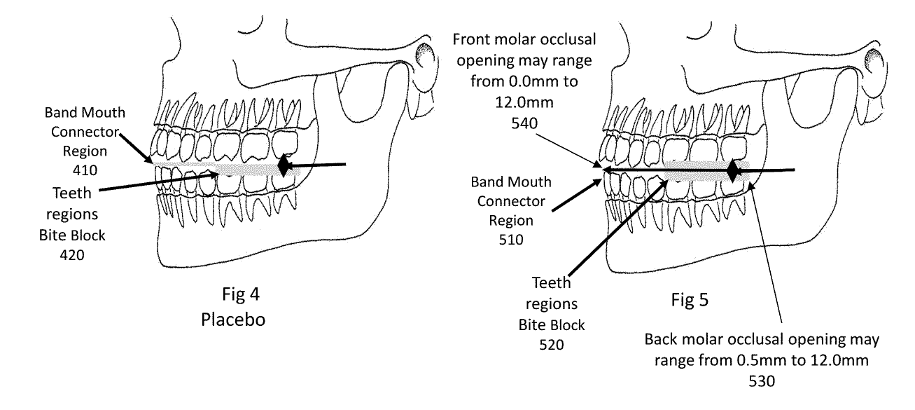

FIG. 4 illustrates a partial cross-sectional view of a placebo mouthpiece in the mouth of a person. The placebo mouthpiece of FIG. 4 also includes a band mouth connector region 410 and teeth regions in a bite block 420. The placebo mouthpiece of FIG. 4 has no incisal opening. As such, a placebo mouthpiece minimizes an amount of air that may pass through the teeth opening when an athlete breathes through their mouth. Such a placebo mouthpiece may be used as a control when experiments that identify an optimal incisal opening for an individual are performed.

FIG. 5 illustrates a partial cross-sectional view of a mouthpiece in the mouth of a person. FIG. 5 also includes a band mouth connector region 510 and a bite block with teeth regions 520. FIG. 5 illustrates an incisal opening of 3 millimeters (mm), however, this can range from 0.5 mm to 6.0 mm or more. Here again the optimized incisal opening helps increase the oxygenation of the brain and muscles of an athlete during and after the athlete performs an activity. Note that the mouthpiece of FIG. 5 controls a back molar occlusal opening 530 that may range from 0.5 mm to 12.0 mm, for example. The mouthpiece of FIG. 5 may also help control a front molar occlusal opening 540 that may range from 0.5 mm to 12.0 mm.

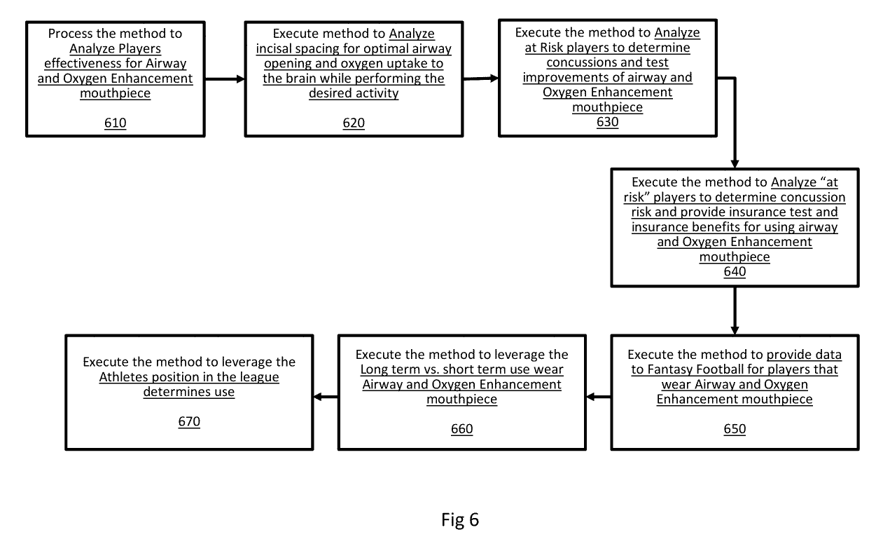

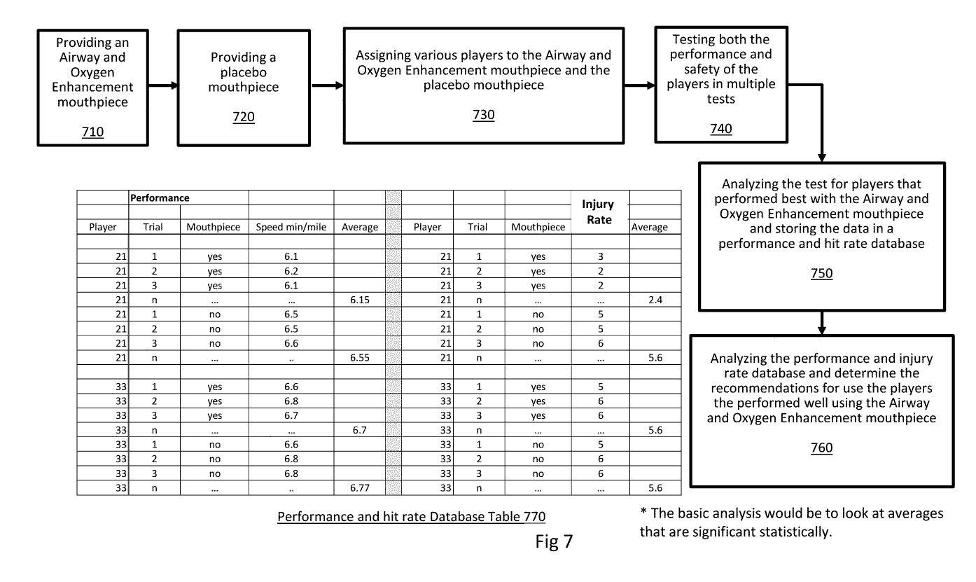

FIG. 6 illustrates an exemplary flow chart of steps that are consistent with a method of the present disclosure. FIG. 6 begins with step 610 where player's activities are analyzed when identifying the effectiveness of an airway and oxygen enhancement mouthpiece. This first step 610 may include a number of exercises may be performed by an athlete when the athlete's performance is being measured. For example, the time a player takes to run 100 yards may be measured. This step may include the athlete wearing a placebo mouthpiece to set a baseline that may be representative of a worst case breathing efficiency for the athlete when performing an activity. Step 610 may also include measuring parametric information, such as blood oxygenation levels before, during, and after the activity via oxygen sensors or tests.

Step 620 of FIG. 6 is where an incisal opening (gap size) may be varied when an athlete performs the same activity with different mouthpieces. Step 620 of FIG. 6 may be used to identify an optimal or preferred incisal opening for a particular athlete. For example, if an athlete always runs 100 yards faster with an incisal opening of 4 mm, that athlete's optimal incisal opening may be identified as being 4 mm.

Step 630 of FIG. 6 is where at the performance of at-risk players may be monitored when identifying whether specific risk factors may be analyzed for improvements. For example, a player may be monitored during a sporting event after an optimal incisal opening is identified for a player that has a high risk of concussions. When blood oxygenation levels are monitored for the at-risk player, a blood oxygenation level dropping below a threshold level may indicate that the player is at an increased risk of receiving a concussion.

Step 640 of FIG. 6 is where data collected from at-risk players may be further analyzed when identifying insurance tests and insurance benefits that may be provided to the at-risk players. This additional analysis may identify a size of airway and oxygen enhancement mouthpiece that corresponds to a lower injury rate even when that particular mouthpiece size does not provide maximum performance. This additional analysis may be also combined with other data when demonstrating to insurance companies that method consistent with the present disclosure may be used to mitigate injury risk when insurance rates are negotiated or established. Other benefits that this additional analysis may provide include correlating blood oxygen levels or other physiological parametric data to risk of concussion after an impact to an athlete's head.

Step 650 of FIG. 6 is where athlete performance data may be provided to fantasy football players that wear airway and oxygen enhancement mouthpieces. Such data may then be used by those fantasy football players when methods consistent with the present disclosure are performed.

Step 660 of FIG. 6 is where the short and the long term wearing of an airway and oxygen enhancement mouthpiece may be monitored. In such instances the thickness of a mouthpiece or the incisal opening of an athlete may be monitored when identifying that an old mouthpiece should be replaced with a new mouthpiece.

Finally, step 670 of FIG. 6 is where an athlete's position in a league maybe conditional upon using a mouthpiece that is optimized for the athlete. As such, a player that is monitored frequently according to method of the present disclosure may be authorized to play more frequently.

FIG. 7 illustrates an exemplary flow chart of a method for analyzing the effectiveness of players using a mouthpiece versus those same players using a placebo. FIG. 7 also includes tabulated results 770 of the players performing athletic tests when they used a mouthpiece as compared to a placebo. A first step, 710, of the flow chart of FIG. 7 is where an airway and oxygen enhancement mouthpiece with an incisal opening of 3 mm. In step 710, various players may perform athletic actions while their performance is measured.

Step 720 of FIG. 7 is where the players may be provided with a placebo mouthpiece, here again the players perform the same athletic actions while their performance is measured.

Next, in step 730 of FIG. 7, various players are assigned a mouthpiece with a 0.5 mm to 6.0 mm incisal opening, for example, where other players are assigned a placebo mouthpiece with an incisal opening of 0 mm. Step 740 of FIG. 7 is where the performance and safety of the 0.5-6.0 mm mouthpiece and the 0 mm mouthpiece are measured. Then, in step 750 of the flow chart of FIG. 7, the player test data is analyzed and stored in a database. Step 750 of FIG. 7 may also identify and store injury rates. Injury rate data may correspond to an event where a player sustains a concussion or injury that requires them to be removed from the competition, training, or practice.

Step 760 of FIG. 7 is where the performance and injury rate data collected in step 750 of FIG. 7 may be further analyzed when identifying recommendations for the players. Such analysis may review averages of data from a plurality of tests when identifying statistically significant data.

The tabulated data 770 of FIG. 7 may be data collected during tests performed according to the flow chart of FIG. 7. Note that the table of FIG. 7 cross references a player number with a trial (test) number, identifies whether the player was wearing an airway and oxygen enhancing mouthpiece (yes) or a placebo (no), records a rate of running speed of the players in minutes/mile, and includes an average speed for a given player number over a different trial (test) numbers. One portion of the table of FIG. 7 includes a an injury rate number. Note that the tabulated data 770 identifies that generally players ran faster on average when using a an airway and oxygen enhancement mouthpiece as compared to a placebo mouthpiece. For example, player 21 had an average running speed of 6.15 miles/min when using a an airway enhancement mouthpiece as compared to an average running speed of 6.55 miles/min when using a placebo mouthpiece.

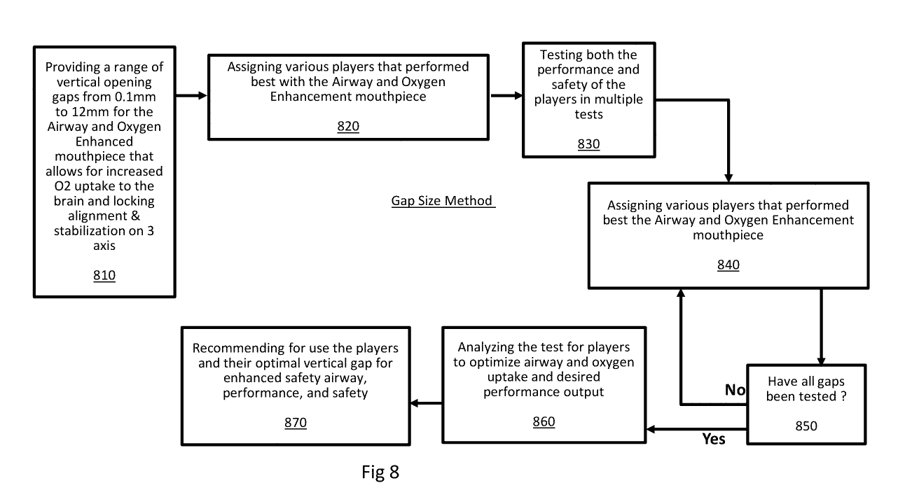

FIG. 8 illustrates an exemplary flow chart of a method that identifies optimal incisal openings for an athlete. Step 810 of FIG. 8 is where different mouthpieces are provided to an athlete, where each different mouthpiece has a different incisal opening. Note that in these experiments the incisal opening and not a gap between the molars is a critically controlled dimension ranging from 0.1 mm to 12.0 mm.

Step 820 of FIG. 8 is where players that performed best with a 0.5-6.0 mm incisal opening are assigned a mouthpiece that provides their custom opening for their preferred or optimal desired output tests. Next, in step 830 of FIG. 8, data relating to both safety factors and performance factors may be collected when the players performed tests.

Step 840 of FIG. 8 is where players may be assigned a mouthpiece that has a different incisal opening and thickness. In step 840, the players may perform the same tests as those performed in the step 830 of FIG. 8. Then, step 850 of FIG. 8 may identify whether all mouthpieces sizes from 0.5 mm to 8.0 mm have been tested, when no, the flow chart moves back to step 840 of FIG. 8 where a next incremental size mouthpiece is tested. After all mouthpiece sizes have been tested, the flow chart moves from step 850 to step 860 of FIG. 8.

In step 860 of FIG. 8, all of the data collected may be analyzed to identify an incisal gap that corresponds to a best performance for a given player. Finally, in step 870 of FIG. 8, each player may be provided with a recommended preferred sized airway and oxygen enhancement mouthpiece.

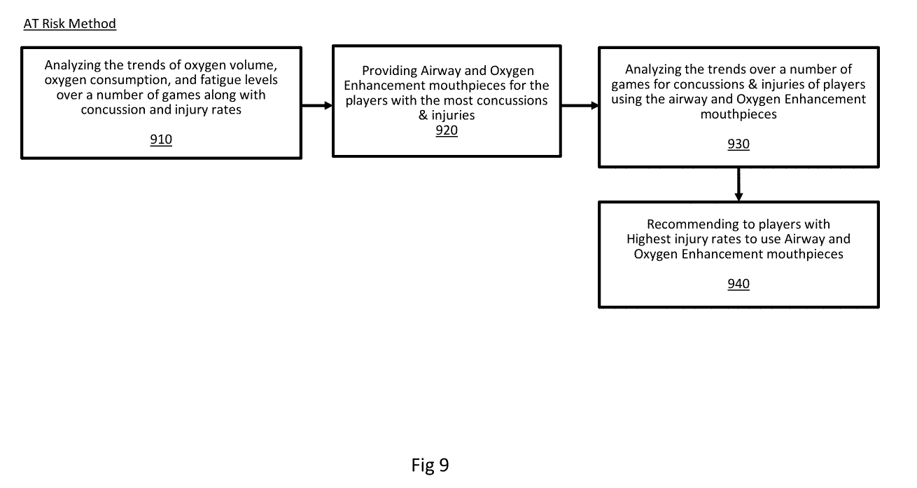

FIG. 9 illustrates an exemplary method for cross referencing incisal openings with player risk factors. Step 910 of FIG. 9 includes analyzing player data collected over a series of games for trends that may be associated with a risk factor. Step 910 of FIG. 9 may include cross referencing injury rate information with impact level, oxygen volume, oxygen consumption, and fatigue level information. Such risk factors may be associated with a number of hits, physical trauma, and with higher impact levels.

Step 920 of FIG. 9, players with the concussion or injury rates may be provided with an airway and oxygen enhancement mouthpiece or with an airway and oxygen enhancement mouthpiece of a different size. Then, in step 930 of FIG. 9, data collected may be analyzed when identifying a size of airway and oxygen enhancement mouthpiece that provides best protection to a player.

Finally, in step 940 of FIG. 9, each player may receive a recommendation identifying a size of airway and oxygen enhancement mouthpiece that provides the best protection and/or performance.

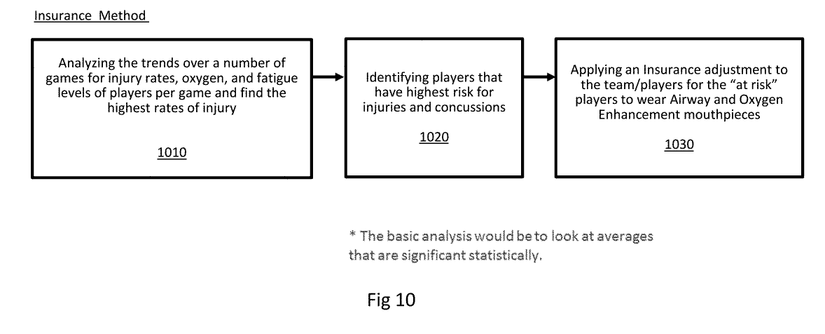

FIG. 10 illustrates an exemplary insurance method consistent with the present disclosure. The step 1010 of FIG. 10 is where data collected over a period of time may be analyzed for trends by cross referencing oxygen uptake, fatigue, and injury rates. Next, in step 1020 of FIG. 10, players with a high risk of injury (concussions) may be identified from the data. Finally, in step 1030 of FIG. 10, an insurance adjustment may be applied to “at-risk” players.

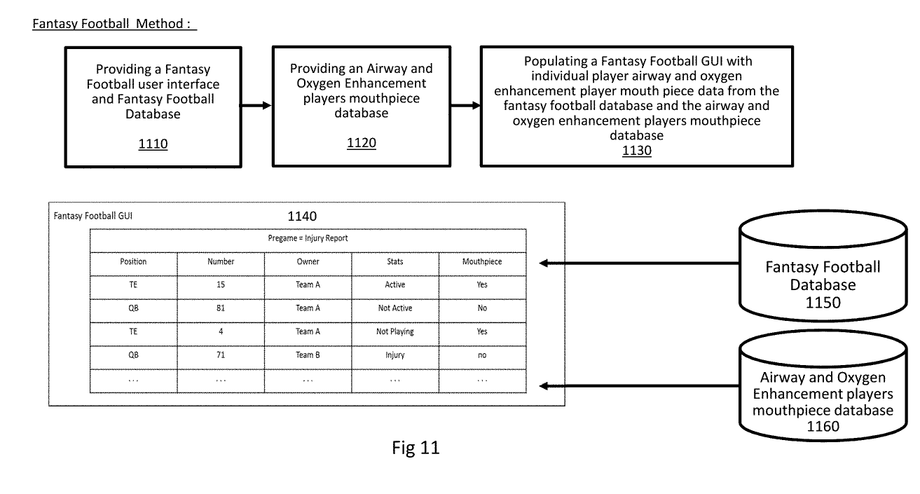

FIG. 11 illustrates an exemplary method of the present disclosure applied to fantasy football. The step 1110 of FIG. 11 is where a user interface and access to a database may be provided to players. Next, in step 1120 of FIG. 11, players of fantasy football may be provided with access to an airway and oxygen enhancement mouthpiece player database. In step 1130 of FIG. 11, a graphical user interface (GUI) may be provided to fantasy football players on a computing device. In this third step of FIG. 11, fantasy football players may use the GUI when reviewing data stored in an airway and oxygen enhancement database and in a fantasy football database.

FIG. 11 also includes tabulated data 1140 that may have been retrieved from a fantasy football database and from an airway and oxygen enhancement database when populating a information in a table 1140 that may be displayed in a graphical user interface (GUI). The tabulated data 1140 of FIG. 11 cross references player position (i.e. tail end (TE) and quarterback (QB)), with a player number, with a team owner, with statistical data (active/not active), and with whether a player is using an airway and oxygen enhancement mouthpiece. Data related to identifying optimal mouthpieces an player data may be stored in a fantasy football database 1150 and in an airway an doxygent enhancement players mouthpiece data base 1160 of FIG. 11.

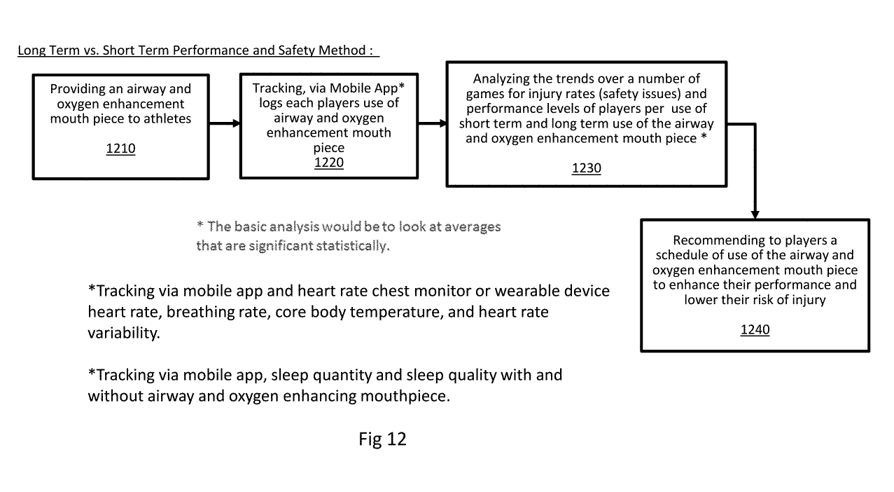

FIG. 12 illustrates an exemplary method from monitoring long term versus short term performance and safety metrics relating to athletes wearing airway and oxygen enhancement mouthpieces. A step 1210 in the flow chart of FIG. 12 is where airway and oxygen enhancement mouthpieces are provided to athletes. Next, in step 1220 of FIG. 12, a mobile application executing on a mobile electronic device collects (logs) player performance and safety data when various player are wearing airway and oxygen enhancement mouthpieces.

The step 1230 of FIG. 12 relates to analyzing the collected player data over a number of games. The data collected may include hit rates or injury rates over a short time period (or number of games) and over a longer time period (or number of games).

The step 1240 of FIG. 12 is where player data collected over the short time period and over the longer time period is used when making recommendations to players regarding a size of an airway and oxygen enhancement mouthpiece that provides specific players with enhances performance and safety metrics. Performance metrics evaluated over time may include running speed may include running speed, impact level, blood oxygen levels, breathing rates, core body temperature, heart rates for a given level of exertion or work load, and variability in heart rate. Safety metrics evaluated over time may include hit rates and injury rates. The method may also include monitoring via the mobile application sleep quality after airway and oxygen enhancement mouthpieces have been versus when an airway and oxygen enhancement mouthpiece was not used.

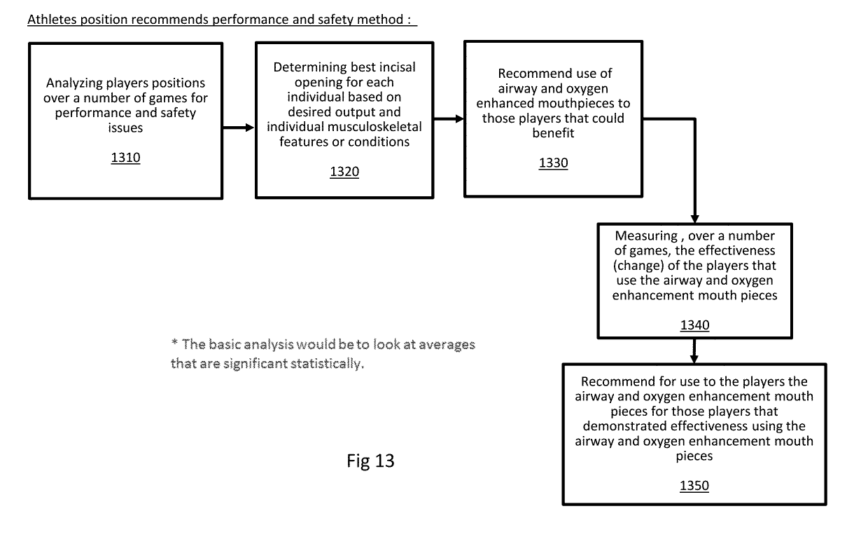

FIG. 13 illustrates an exemplary method of the present disclosure where performance and safety metrics are collected when athletes play different positions in a game or series of games. Step 1310 in FIG. 13 is where data collected from players playing different positions is analyzed. Next, in step 1320 of FIG. 13, players that could benefit from using airway and oxygen enhancement mouthpieces are identified using statistical data analysis. Then, in step 1330 of FIG. 13, players may receive recommendations identifying sizes of airway and oxygen enhancement mouthpieces that could benefit those players the most.

The step 1340 of FIG. 13 may then identify the effectiveness of airway and oxygen enhancement mouthpieces over a number of games, where a higher effective mouthpiece size may be associated with higher performance metrics or with higher safety metrics. Finally, in step 1350 of the flow chart of FIG. 13, players could receive recommendations identifying a size of airway and oxygen enhancement mouthpiece that is most effective for each individual player on a team.

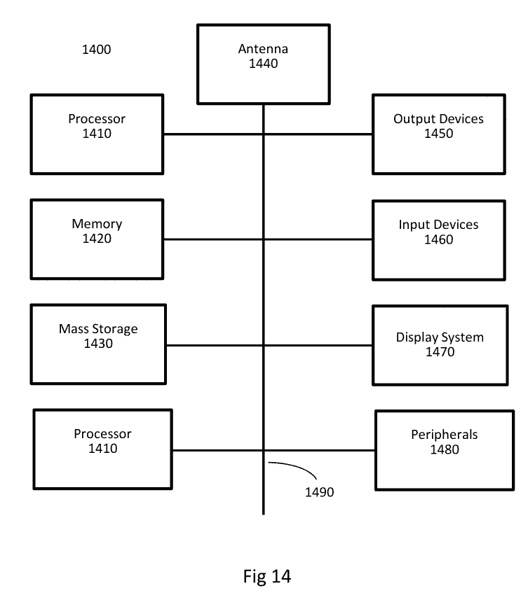

FIG. 14 is a block diagram of a device for implementing the present technology. FIG. 14 illustrates an exemplary computing system 1400 that may be used to implement a computing device for use with the present technology. System 1400 of FIG. 14 may be implemented in the contexts of the likes of clients and servers. The computing system 1400 of FIG. 14 includes one or more processors 1410 and memory 1420. Main memory 1420 may store, in part, instructions and data for execution by processor 1410. Main memory 1420 can store the executable code when in operation. The system 1400 of FIG. 14 further includes mass storage 1430, which may include resident mass storage and portable storage, antenna 1440, output devices 1450, user input devices 1460, a display system 1470, and peripheral devices 1480.

The components shown in FIG. 14 are depicted as being connected via a single bus 1490. However, the components may be connected through one or more data transport means. For example, processor unit 1410 and main memory 1420 may be connected via a local microprocessor bus, and the storage 1430, peripheral device(s) 1480, and display system 1470 may be connected via one or more input/output (I/O) buses.

Mass storage device 1430, which may include mass storage implemented with a magnetic disk drive or an optical disk drive, or be a portable storage device. Mass storage device 1430 can store the system software for implementing embodiments of the present invention for purposes of loading that software into main memory 1420.

In certain instances mass storage 1430 may include a portable storage device, such as a floppy disk, compact disk, a Digital video disc, or a USB data storage device. The system software for implementing embodiments of the present invention may be stored on such a portable medium and input to the computer system 1400 via the portable storage device.

Antenna 1440 may include one or more antennas for communicating wirelessly with another device. Antenna 1440 may be used, for example, to communicate wirelessly via Wi-Fi, Bluetooth, with a cellular network, or with other wireless protocols and systems. The one or more antennas may be controlled by a processor 1410, which may include a controller, to transmit and receive wireless signals. For example, processor 1410 executes programs stored in memory 1420 to control antenna 1440 transmit a wireless signal to a cellular network and receive a wireless signal from a cellular network.

The system 1400 as shown in FIG. 14 includes output devices 1450 and input devices 1460. Examples of suitable output devices include speakers, printers, network interfaces, and monitors. Input devices 1460 may include a touch screen, microphone, accelerometers, a camera, and other devices. Input devices 1460 may also include an alpha-numeric keypad, such as a keyboard, for inputting alpha-numeric and other information, or a pointing device, such as a mouse, a trackball, stylus, or cursor direction keys.

Display system 1470 may include a liquid crystal display (LCD), LED display, a plasma display, or be another suitable display device. Display system 1470 receives textual and graphical information, and processes the information for output to the display device.

Peripherals 1480 may include any type of computer support device to add additional functionality to the computer system. For example, peripheral device(s) 1480 may include a modem or a router.

The components contained in the computer system 1400 of FIG. 14 are those typically found in computing system, such as but not limited to a desktop computer, laptop computer, notebook computer, netbook computer, tablet computer, smart phone, personal data assistant (PDA), or other computer that may be suitable for use with embodiments of the present invention and are intended to represent a broad category of such computer components that are well known in the art. Thus, the computer system 1400 of FIG. 14 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

The various methods may be performed by software operating in conjunction with hardware. For example, instructions executed by a processor, the instructions otherwise stored in a non-transitory computer readable medium such as memory. Various interfaces may be implemented—both communications and interface. One skilled in the art will appreciate the various requisite components of a mobile device and integration of the same with one or more of the foregoing figures and/or descriptions.

While various embodiments have been described above, it should be understood that they have been presented by way of example only, and not limitation. The description is not intended to limit the scope of the presently claimed invention or to limit the scope of embodiments of the presently claimed invention. The present descriptions are intended to cover alternatives, modifications, and equivalents consistent with the spirit and scope of the disclosure.

What is claimed is:

1. A method for selecting an optimal mouthpiece for a wearer, the method comprising:
collecting performance data for each of a plurality of different mouthpieces worn by the wearer during performance of an activity over time, wherein each mouthpiece has a different vertical opening and occlusal thickness, the collected data stored in memory;
analyzing the collected performance data; and
identifying, based on the analysis of the collected performance data, a preferred mouthpiece of the plurality of different mouthpieces.

2. The method of claim 1, wherein each of the different vertical openings and occlusal thicknesses adjust a front molar occlusal opening and a back molar occlusal opening when the respective mouthpiece is worn.

3. The method of claim 1, wherein the performance data comprises oxygen levels in blood of the wearer.

4. The method of claim 1, wherein the performance data comprises a running speed of the wearer.

5. The method of claim 1, wherein the wearer is an athlete and the activity is a sport.

6. The method of claim 1, wherein the performance data comprises at least one of an impact level, breathing rates, core body temperature, heart rates for a given level of exertion or work load.

7. The method of claim 1, wherein the performance data comprises at least one of a variability in a heart rate of the person, an impact rate, and injury rate.

8. A non-transitory computer readable storage medium for executing a method for selecting an optimal mouthpiece for an athlete to wear when participating in a contact sport, the method comprising:
collecting performance data for each of a plurality of different mouthpieces worn by the wearer during performance of an activity over time, wherein each mouthpiece has a different vertical opening and occlusal thickness, the collected data stored in memory;
analyzing the collected performance data; and
identifying, based on the analysis of the collected performance data, a preferred mouthpiece of the plurality of different mouthpieces.

9. The non-transitory computer readable storage medium of claim 8, wherein each of the different vertical openings and occlusal thicknesses adjust a front molar occlusal opening and a back molar occlusal opening of the person when the respective mouthpiece is worn.

10. The non-transitory computer readable storage medium of claim 8, wherein the performance data comprises oxygen levels in blood of the wearer.

11. The non-transitory computer readable storage medium of claim 8, wherein the performance data comprises a running speed of the wearer.

12. The non-transitory computer readable storage medium of claim 8, wherein the wearer is an athlete and the activity is a sport.

13. The non-transitory computer readable storage medium of claim 8, wherein the performance data comprises at least one of an impact level, breathing rates, core body temperature, heart rates for a given level of exertion or work load.

14. The non-transitory computer readable storage medium of claim 8, wherein the performance data comprises at least one of a variability in a heart rate of the person, an impact rate, and injury rate.

15. A system for selecting an optimal mouthpiece for a wearer, the system comprising:
a plurality of different mouthpieces, each mouthpiece having a different thickness corresponding to a different incisal opening in a mouth of the wearer;
one or more sensors that collect data regarding the wearer of the plurality of different mouthpieces during performance of an activity over time,
memory that stores the collected data; and
a processor that executes instructions stored in memory, wherein execution of the instructions by the processor identifies a preferred mouthpiece from the plurality of mouthpieces, the selection based on analysis of the collected performance data.

16. The system of claim 15, wherein each of the different vertical openings and occlusal thicknesses adjust a front molar occlusal opening and a back molar occlusal opening when the respective mouthpiece is worn.

17. The system of claim 15, wherein the performance data comprises oxygen levels in blood of the wearer.

18. The system of claim 15, wherein the performance data comprises a running speed of the wearer.

19. The system of claim 15, wherein the wearer is an athlete and the activity is a sport.

20. The system of claim 15, wherein the performance data comprises at least one of an impact level, breathing rates, core body temperature, heart rates for a given level of exertion or work load.
